(19) United States
(12) Patent Application Publication
(10) Pub. No.: US 2020/0060819 A1
(43) Pub. Date: Feb. 27, 2020

(54) AORTIC VALVE EVALUATION ASSISTING JIG

(71) Applicant: SUN MEDICAL TECHNOLOGY RESEARCH CORPORATION, Nagano (JP)

(72) Inventor: Kenji YAMAZAKI, Hokkaido (JP)

(21) Appl. No.: 16/547,485

(22) Filed: Aug. 21, 2019

(30) Foreign Application Priority Data
Aug. 22, 2018 (JP) 2018-155718

(51) Int. Cl. A61F 2/24

(52) U.S. Cl. CPC A61F 2/2427; A61B 2017/00243; A61F 2/2418

(57) ABSTRACT

Provided is an aortic valve evaluation assisting jig which includes a partition wall member which partitions an aortic duct inner space into a space on an atmosphere open side and a space on an aortic valve side in a state where at least a part of the partition wall member is disposed in the aortic duct inner space, the partition wall member has: a seal portion which seals an evaluation liquid in a state where the seal portion is brought into contact with the aortic wall of an aortic duct inner space side; and a filling port through which the evaluation liquid is filled into the space on the aortic valve side in the aortic duct inner space.

### BACKGROUND OF THE INVENTION

### 1. Field of the Invention

The present invention relates to an aortic valve evaluation assisting jig.

### 2. Description of the Related Art

As a method for curing a disease relating to an aortic valve (aortic stenosis, aortic insufficiency or the like), conventionally, “valve replacement surgery” has been introduced and practiced.

Valve replacement surgery is an operation where a patient's own valve (the description of the valve being made hereinafter by focusing on an aortic valve in this specification) is replaced with a prosthetic cardiac valve such as a mechanical valve or a bioprosthetic valve. Thanks to this valve replacement surgery, the lives of many people have been saved.

However, valve replacement surgery also has a drawback. A mechanical valve is made of an artificial material and hence, during the use of the mechanical valve, blood is liable to adhere to the mechanical valve and hence, thrombus is liable to occur. To overcome such a drawback, a patient has to keep taking an anticoagulant such as Warfarin for preventing thrombus, and such compulsory dosing of the anticoagulant imposes a large strain on the patient. In the case where a dosage of an anticoagulant is excessively large, when a patient suffers from an injury, it is not easy to stop bleeding of blood thus giving rise to a risk of hemorrhage. In view of the above, it is necessary to take a measure such as constant monitoring of a state of blood and the adjustment of a dosage of an anticoagulant when necessary.

On the other hand, when a patient's own valve is replaced with a bioprosthetic valve, it is unnecessary for the patient to take an anticoagulant. However, the bioprosthetic valve deteriorates with the lapse of time and hence, for example, hardening of the bioprosthetic valve occurs. Accordingly, the bioprosthetic valve is less than optimal from a viewpoint of durability.

In view of the drawbacks of such valve replacement surgery, recently, treatment by “valvuloplasty” which does not use a prosthetic cardiac valve has been proposed.

Valvuloplasty is an operation where an aortic valve is reconstructed by repairing the valve by making use of a patient's own existing valve (for example, see Kenji Minakata, M D et al., “Is repair of aortic valve regurgitation a safe alternative to valve replacement?”, The Journal of Thoracic and Cardiovascular Surgery, Vol. 127, Number 3, pp. 645-653 (non-patent literature 1)). Valvuloplasty is an operation where an existing valve is repaired by suitably changing a shape, a structure, a size or the like of the valve corresponding to a state of the existing valve so as to restore original functions which the valve should possess or to impart the valve functions substantially equal to original functions which the valve should possess.

Such valvuloplasty uses neither a mechanical valve nor a bioprosthetic valve. Accordingly, a disease relating to an aortic valve can be cured without causing the above-mentioned drawbacks including a drawback brought about by taking an anticoagulant or a drawback on durability of bioprosthetic valve.

However, valvuloplasty of an aortic valve is still less than optimal because valvuloplasty has a drawback that there is no means for evaluating a valve repaired by valvuloplasty. This drawback is described more hereinafter.

### SUMMARY OF INVENTION

### (1) Necessity of Carrying Out Evaluation of Valve in Valvuloplasty

A mechanical valve is designed so as to exhibit functions which the valve should possess. On the other hand, with respect to a bioprosthetic valve, an aorta is incised (cut) and, thereafter, a size of an aortic annular is measured, and a prosthetic cardiac valve having a size which conforms to a patient is embedded in a human body by suturing. In both cases, a substitute valve (prosthetic cardiac valve) which is in a completed state as a so-called part is embedded in the human body. Accordingly, by performing an operation for embedding a prosthetic cardiac valve having an appropriate size, the prosthetic cardiac valve can substantially realize original functions which an aortic valve should possess with almost certainty.

In this specification, “functions which an aortic valve should possess” means, for example, functions such as “a function of opening the valve and supplying blood without causing an excessive pressure loss when a pressure in a left ventricle is larger than a pressure in an aortic duct (when “a pressure in a forward direction” which is a direction that blood flows is applied)”, and “a function of closing the valve and preventing regurgitation of the blood when a pressure in an aortic duct in the vicinity of aortic valve is larger than a pressure in the left ventricle (when “a pressure in a reverse direction” is applied) to the contrary”. However, “functions which an aortic valve should possess” are not limited to the functions described above.

On the other hand, valvuloplasty is an operation for repairing the valve while making use of a patient's own existing valve having a defect. Accordingly, after the operation is performed, it is necessary to evaluate whether or not the valve is formed as expected or intended or to evaluate whether or not the valve has succeeded in recovering functions substantially equal to original functions which the patient's own existing valve should possess (evaluation of the valve).

### (2) Difficulty in Aortic Valvuloplasty

In valvuloplasty of aorta (aortic valvuloplasty), it is possible to provisionally perform simple confirmation of a valve by visually observing a shape or the like of the valve in a state where the valve is exposed to atmosphere (an atmosphere open state) immediately after a valve repairing operation.

However, whether or not the repaired aortic valve can exhibit original functions which the patient's own existing valve should possess in a state where a physiological pressure is applied cannot be actually understood unless heart beat is restarted. Such a circumstance is described in detail hereinafter.

FIG. 9 is a view showing a state where an aortic wall 822 in the vicinity of an aortic valve 810 is incised in aortic valvuloplasty. FIG. 9 shows four chambers which form a heart 800, that is, a left atrium 840, a left ventricle 845, a right atrium 850 and a right ventricle 855, and peripheries of the heart 800 in cross section. Symbol 550 indicates an aortic cross-clamp, and symbol 830 indicates a mitral valve.

Firstly, valvuloplasty of the mitral valve 830 (mitral annuloplasty) is described. Although FIG. 9 shows the case of aortic valvuloplasty, FIG. 9 is also used in the description of mitral annuloplasty by focusing on the mitral valve 830 and an area in the vicinity of the mitral valve 830, aortic cross-clamp 550 and the like shown in FIG. 9.

Although the detailed illustration of mitral annuloplasty is omitted, in mitral annuloplasty, the evaluation of a valve during an operation under a cardiac arrest can be performed. During the operation, the aorta 820 is shut off (forward flow and backward flow of blood being shut off) by the aortic cross-clamp 550. However, in this state, by inserting a tube between an anterior cusp and a posterior cusp of the mitral valve 830 from an opening portion formed in a left atrium wall and filling and charging an evaluation liquid (physiological saline in this case) into the left ventricle through the tube, a pressure in the left ventricle can be increased. In this case, a pressure in a reverse direction is applied to the mitral valve 830, and acts so as to close the anterior cusp and the posterior cusp of the mitral valve 830 (thereafter, the tube being released). By bringing about such a circumstance, a joining state of the mitral valve 830, the presence and non-presence of regurgitation and the like can be confirmed and hence, the evaluation of the valve can be performed. Further, such an operation can be relatively easily performed repeatedly during the operation.

On the other hand, as shown in FIG. 9, in aortic valvuloplasty, the aortic valve 810 is disposed at the position in a forward direction as viewed from the inside of the left ventricle 845. Accordingly, even if a method substantially equal to the above-mentioned evaluation method of the mitral valve 830 is adopted, an evaluation liquid leaks from the inside of the left ventricle 845 and hence, the evaluation of the valve cannot be performed.

Accordingly, in aortic valvuloplasty, there is no other way to evaluate the valve than by the method where, after the valve is formed, the incised (cut) aortic wall 822 (not shown in FIG. 9) is closed by suturing, the interruption of aorta is released and heart beat is restarted, and the movement of the valve, the flow of blood and the like are indirectly observed from the outside of the heart 800 by ultrasonic diagnostic equipment (echocardiography).

If it is determined that there is a defect in forming the valve and there exists residual regurgitation or the like, it is necessary to perform the steps again where the aorta 820 is shut off, the cardiac arrest is reintroduced, and the aorta 820 closed by anastomosis previous time is incised again. In this manner, in a situation where it is necessary to perform a cardiac arrest plural times, not only a drawback that efficiency of an operation is lowered but also a drawback that a damage to a cardiac muscle and the invasion to a patient are increased occurs. These are factors which make a surgeon hesitate to adopt aortic valvuloplasty.

As has been described above, conventionally, in aortic valvuloplasty, there has been no means which evaluates an aortic valve during an operation under a cardiac arrest (see also a Chapter “Discussion” in non-patent literature 1). This lack of means which evaluates an aortic valve is the largest factor which makes it difficult for a surgeon to perform aortic valvuloplasty, and also becomes a factor which holds back the popularization of aortic valvuloplasty.

The present invention has been made in view of the above-mentioned circumstances, and it is an object of the present invention to provide an aortic valve evaluation assisting jig which assists the evaluation of a valve such that the evaluation of the valve can be performed even under a cardiac arrest for aortic valvuloplasty.

[1] According to an aspect of the present invention, there is provided an aortic valve evaluation assisting jig which assists evaluation of an aortic valve formed by aortic valvuloplasty at the time of performing the evaluation of the aortic valve, wherein the aortic valve evaluation assisting jig includes a partition wall member which partitions an aortic duct inner space into a space on an atmosphere open side and a space on an aortic valve side in a state where at least a part of the partition wall member is disposed in the aortic duct inner space (inside of the aortic duct), and the partition wall member has: a partition wall body portion; a seal portion which seals an evaluation liquid in a state where the seal portion is brought into contact with the aortic wall of an aortic duct inner space side; and a filling port through which the evaluation liquid is filled into the space on the aortic valve side in the aortic duct inner space.
[2] In the aortic valve evaluation assisting jig of the present invention, it is preferable that an observation window formed of a transparent member through which the space on the aortic valve side in the aortic duct inner space can be viewed from the space on the atmosphere open side in a see-through manner be disposed on the partition wall body portion.
[3] In the aortic valve evaluation assisting jig of the present invention, it is preferable that a camera which images a mode of the space on the aortic valve side in the aortic duct inner space be disposed on the partition wall body portion.
[4] In the aortic valve evaluation assisting jig of the present invention, it is preferable that the partition wall member be formed in an approximately cup shape and be formed of: a cup bottom portion having a flat plate-like shape; and a tapered portion formed such that an outer diameter is increased from an outer peripheral edge of the cup bottom portion toward a cup upper edge having an approximately circular shape, the filling port be formed in the cup bottom portion, and the seal portion be formed of the tapered portion.
[5] In the aortic valve evaluation assisting jig of the present invention, it is preferable that the partition wall member be formed in a bottomed approximately cylindrical shape and be formed of: a cylindrical barrel portion having an opening portion on one side; and a cylinder bottom portion connected to the other side of the cylindrical barrel portion, wherein

a flange-shaped upper edge be formed at the cylindrical barrel portion on an opening portion side,

the cylindrical barrel portion have: a tapered portion which has an outer diameter increasing from an outer peripheral edge of the cylinder bottom portion toward the opening portion side; and a groove portion which is disposed between an uppermost end of the tapered portion and the flange-shaped upper edge and has a diameter smaller than an outer diameter of the uppermost end of the tapered portion and smaller than an outer diameter of the flange-shaped upper edge,

the filling port be formed in the cylinder bottom portion, and

the seal portion be formed of the groove portion.

[6] It is preferable that the aortic valve evaluation assisting jig of the present invention further include a hollow pipe in which a liquid flow passage is formed, and one end of the hollow pipe be connected to the filling port formed in the partition wall member.
[7] In the aortic valve evaluation assisting jig of the present invention, it is preferable that the seal portion be formed using a resin, or an outside of the seal portion be covered by a resin-made member.

According to the aortic valve evaluation assisting jig of the present invention, the partition wall member has the filling port. Accordingly, by filling an evaluation liquid (a myocardial protection liquid or the like) from the filling port into the space on the aortic valve side in the aortic duct inner space, the space can be filled with the evaluation liquid.

Further, the partition wall member of the aortic valve evaluation assisting jig partitions the space in the aortic duct inner space into the space on the atmosphere open side and the space on the aortic valve side in the aortic duct inner space in a state where at least a part of the partition wall member is disposed in the aortic duct inner space, and the partition wall member has the seal portion which seals the evaluation liquid in a state where the seal portion is brought into contact with the aortic wall of an aortic duct inner space side.

Accordingly, even if the space on one side of the partition wall member is open to atmosphere, in the space on the other side of the partition wall member, an internal pressure of a liquid in the space on an aortic valve side in the aortic duct inner space can be increased.

That is, a pressure in a reverse direction can be applied to the aortic valve.

Accordingly, by suitably adjusting a filling pressure of the evaluation liquid, it is possible to bring aortic cusps of the aorta into contact with each other by applying an intended pressure (for example, a physiological pressure) to the aortic valve. That is, whether or not the repaired aortic valve can exhibit original functions which the valve should possess can be evaluated not under an atmosphere opened state but under a state where an intended pressure is applied.

The observation of the aortic valve can be performed by directly viewing through an observation window formed of a transparent member disposed on the partition wall body portion described later, by viewing through a camera disposed on the partition wall body portion, or by viewing using an image of an ultrasonic diagnostic equipment (echocardiography). The evaluation of the valve is performed based on a result of the observation exemplified above.

Accordingly, an aortic valve evaluation assisting jig of the present invention can assist the evaluation of a valve such that the evaluation of the valve can be performed even under a cardiac arrest for aortic valvuloplasty.

### BRIEF DESCRIPTION OF DRAWINGS

FIG. 1A and FIG. 1B are views for describing an aortic valve evaluation assisting jig 1 according to an embodiment 1;

FIG. 2A and FIG. 2B are views for describing an aortic valve evaluation assisting jig 2 according to an embodiment 2;

FIG. 3A and FIG. 3B are views for describing a use example of the aortic valve evaluation assisting jig 2 according to the embodiment 2;

FIG. 4A to FIG. 4C are views for describing the use example of the aortic valve evaluation assisting jig 2 according to the embodiment 2 succeeding FIG. 3A and FIG. 3B;

FIG. 5 is a view for describing a use example of (an example of another patient of) the aortic valve evaluation assisting jig 2 according to the embodiment 2;

FIG. 6A and FIG. 6B are views for describing an aortic valve evaluation assisting jig 3 according to an embodiment 3;

FIG. 7A and FIG. 7B are views for describing a use example of the aortic valve evaluation assisting jig 3 according to the embodiment 3;

FIG. 8A and FIG. 8B are views for describing an aortic valve evaluation assisting jig 4 according to a modification; and

FIG. 9 is a view showing a state where an aortic wall 822 in the vicinity of the aortic valve 810 is incised in aortic valvuloplasty.

### DESCRIPTION OF PREFERRED EMBODIMENTS

Hereinafter, an aortic valve evaluation assisting jig according to the present invention is described based on embodiments shown in drawings. The respective drawings are schematic drawings, and do not necessarily strictly reflect actual sizes.

### Embodiment 1

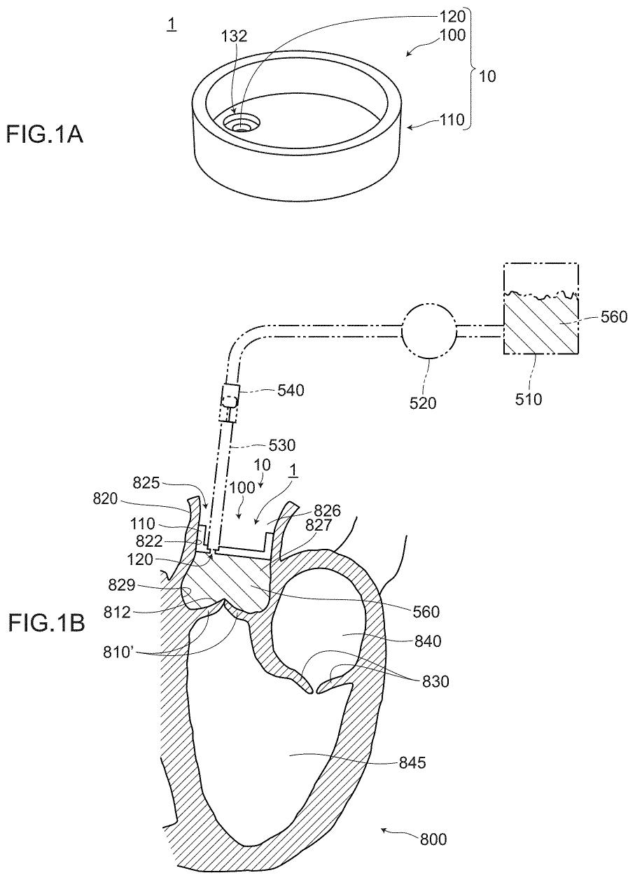

FIG. 1A and FIG. 1B are views for describing an aortic valve evaluation assisting jig 1 according to the embodiment 1. FIG. 1A is a perspective view of the aortic valve evaluation assisting jig 1, and FIG. 1B is a cross-sectional view of a main part showing a use example of the aortic valve evaluation assisting jig 1.

### 1. Configuration of Aortic Valve Evaluation Assisting Jig 1 According to Embodiment 1

As shown in FIG. 1, the aortic valve evaluation assisting jig 1 is an aortic valve evaluation assisting jig which assists the evaluation of an aortic valve 810′ formed in an aortic valvuloplasty at the time of evaluating the aortic valve 810′. The aortic valve evaluation assisting jig 1 includes a partition wall member 10.

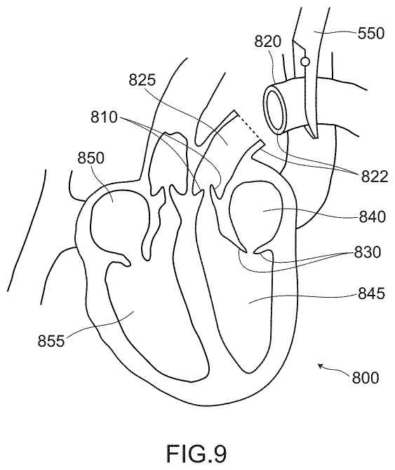

The partition wall member 10 includes at least: a partition wall body portion 100; a seal portion 110; and a filling port 120 for filling an evaluation liquid 560 (see FIG. 1A). The partition wall member 10 partitions the aortic duct inner space 825 into a space on an atmosphere open side 826 and a space on an aortic valve side 827 in a state where at least a part of the partition wall member 10 is disposed in the aortic duct inner space 825 which is the inside of a duct (vessel) of the aorta 820 (see FIG. 1B and FIG. 9).

The partition wall body portion 100 is a main portion by which the space on the atmosphere open side 826 and the space on the aortic valve side 827 are partitioned from each other.

The seal portion 110 is formed in an approximately annular shape along an outer periphery of the partition wall body portion 100. In a state where the partition wall member 10 is wedged into the aortic duct inner space 825, the seal portion 110 is brought into contact with an inner periphery of the duct of the aorta 820. The seal portion 110 seals (liquid sealing) an evaluation liquid 560 in a state where the seal portion 110 is brought into contact with the aortic wall 822 of an aortic duct inner space side. Accordingly, the movement of the liquid from the space on the aortic valve side 827 in the aortic duct inner space 825 to the space on the atmosphere open side 826 in the aortic duct inner space 825 can be prevented (except for a forward flow and backward flow of the liquid through the filling port 120).

The filling port 120 is formed as a discharge port of the liquid when the evaluation liquid 560 is filled in the space on the aortic valve side 827 in the aortic duct inner space 825 (see FIG. 1B).

A fluid flow passage is formed in the partition wall member 10 in a penetrating manner from one side (the atmosphere open side 826 in a state where the partition wall member 10 is disposed in the aortic duct inner space 825) to the other side (aortic valve side 827). In this embodiment, the other side (aortic valve side 827) of the fluid flow passage which is a through hole forms the filling port 120.

By connecting a liquid supply tube 530, a hollow pipe (described later) and the like to a connecting port 132 of a liquid flow passage on one side, and by supplying an evaluation liquid 560 through the liquid supply tube 530, the hollow pipe and the like, the liquid can be discharged from the filling port 120 on the other side of the liquid flow passage through the through hole.

As the evaluation liquid 560, a myocardial protection liquid is used. However, the evaluation liquid 560 is not limited to a myocardial protection liquid. Provided that the aortic valve 810′ can be evaluated without problems physiologically, other liquids (for example, physiological saline and the like) can be also used.

So long as the aortic valve evaluation assisting jig 1 possesses the above-mentioned functions of the partition wall member 10, the aortic valve evaluation assisting jig 1 may be made of any material, and may have any structure, shape and the like. Further, in the aortic valve evaluation assisting jig 1, constitutional elements other than the partition wall member 10 may be added to the partition wall member 10.

### 2. Advantageous Effects Obtained by Aortic Valve Evaluation Assisting Jig 1 According to Embodiment 1

In the aortic valve evaluation assisting jig 1 according to the embodiment 1, the filling port 120 is formed in the partition wall member 10. Accordingly, by filling the evaluation liquid 560 into the space on the aortic valve side 827 in the aortic duct inner space 825 from the filling port 120, the space can be filled with the evaluation liquid 560. In such an operation, air is suitably released through between the seal portion 110 and the aortic wall 822, for example.

The partition wall member 10 of the aortic valve evaluation assisting jig 1 is configured such that the space on the atmosphere open side 826 in the aortic duct inner space 825 and the space on the aortic valve side 827 in the aortic duct inner space 825 are partitioned from each other in a state where at least a portion of the partition wall member 10 is disposed in the aortic duct inner space 825. Further, the partition wall member 10 has the seal portion 110 which seals the evaluation liquid 560 in a state where the seal portion 110 is brought into contact with the aortic wall 822 of the aortic duct inner space side.

Accordingly, even in the case where the space on one side of the partition wall member 10 is in an atmosphere open state, an internal pressure generated by a liquid in the space (including Valsalva sinus) formed on the aortic valve side 827 in the aortic duct inner space 825 can be increased in the space on the other side of the partition wall member 10. That is, it is possible to apply a pressure in a reverse direction to the aortic valve 810′.

Accordingly, by suitably adjusting a filling pressure of the evaluation liquid 560, it is possible to bring aortic cusps 812 of the aorta into contact with each other by applying an intended pressure (for example, a physiological pressure) to the aortic valve 810′. That is, whether or not the repaired aortic valve 810′ can exhibit original functions which the valve should possess can be evaluated not under an atmosphere opened state but under a state where an intended pressure is applied.

The observation of the aortic valve 810′ can be performed by directly viewing through an observation window formed of a transparent member disposed on the partition wall body portion 100 described later (described later in an embodiment 2), by viewing through a camera disposed on the partition wall body portion 100 (described later in “modification” (2)), by viewing using an image of a ultrasonic diagnostic equipment (echocardiography) or the like. The evaluation of the valve is performed based on a result of the observation exemplified above.

As described above, according to the aortic valve evaluation assisting jig 1 of this embodiment 1, the aortic valve evaluation assisting jig 1 can assist the evaluation (the evaluation of the valve) such that the evaluation of the valve (aortic valve 810′) can be performed even under a cardiac arrest in aortic valvuloplasty. As a result, an operation can be realized properly and speedily and hence, a strain imposed on a patient and a surgeon can be reduced.

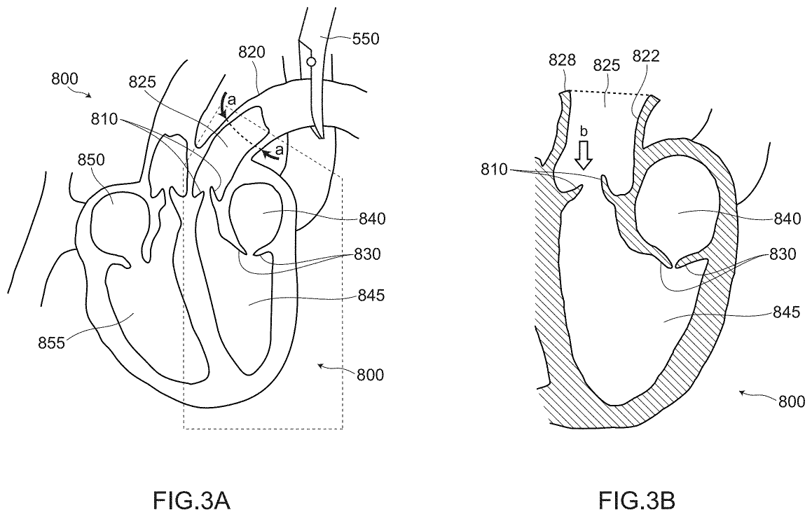

The description has been made heretofore by estimating the evaluation of the aortic valve 810′ after techniques of aortic valvuloplasty are applied to the aortic valve 810′. However, the present invention is not limited to such a case. For example, the aortic valve evaluation assisting jig 1 may be used at the time of evaluating functions as a valve of an aortic valve 810 having a defect (see FIG. 3A described later, not shown in FIG. 1) before techniques of aortic valvuloplasty are applied to the aortic valve 810.

The detailed configuration and a use example of the aortic valve evaluation assisting jig are suitably described in the following chapters, that is, in the embodiment 2 and the embodiment 3.

### Embodiment 2

### 1. Configuration of Aortic Valve Evaluation Assisting Jig 2 According to Embodiment 2

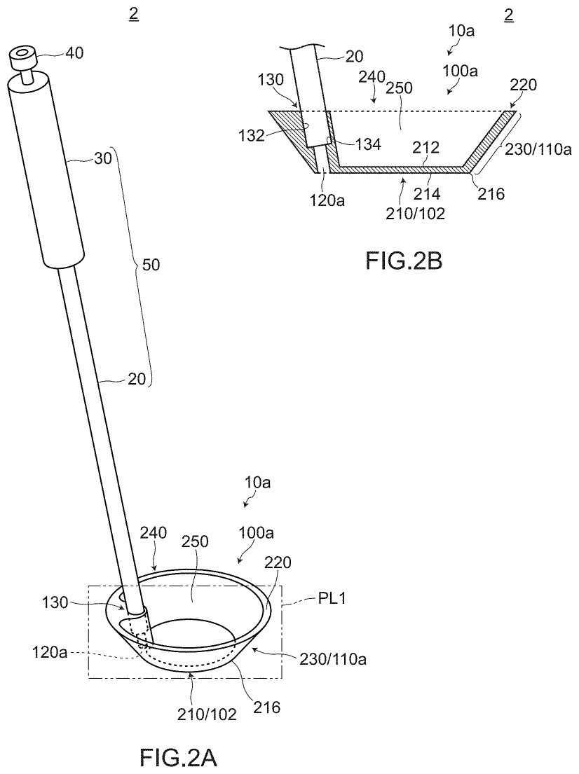

FIG. 2A and FIG. 2B are views for describing an aortic valve evaluation assisting jig 2 according to the embodiment 2. FIG. 2A is a perspective view of the aortic valve evaluation assisting jig 2, and FIG. 2B is a cross-sectional view of a main part of the aortic valve evaluation assisting jig 2 taken along an imaginary cross section PL1 in FIG. 2A. In the embodiment 2, with respect to constitutional elements having basically the same configurations and technical features as the corresponding constitutional elements of the embodiment 1, the same symbols used in the embodiment 1 are used, and the descriptions of these constitutional elements is omitted.

The aortic valve evaluation assisting jig 2 according to the embodiment 2 basically has the same configuration as the aortic valve evaluation assisting jig 1 according to the embodiment 1. However, the aortic valve evaluation assisting jig 1 according to the embodiment 2 differs from the aortic valve evaluation assisting jig 1 according to the embodiment 1 with respect to the specific configuration of the partition wall member.

### (1) Configuration of Partition Wall Member 10a

As shown in FIG. 2A and FIG. 2B, the partition wall member 10a of the embodiment 2 has an approximately cup shape.

The partition wall member 10a has a cup bottom portion 210 and a tapered portion 230 having a conical side surface shape. Since the partition wall member 10a is formed in an approximately cup shape having the tapered portion 230, the partition wall member 10a can be easily wedged into an incised aorta. The partition wall member 10a may be formed using a hard vinyl chloride resin as a main material, for example. As a material for forming the partition wall member 10a, a material which ensures biological safety and has the sterilization specification is adopted.

The cup bottom portion 210 forms a part of the partition wall body portion 100a. The cup bottom portion 210 is formed of a flat plate-like member. For example, the cup bottom portion 210 is formed of a disk-shaped flat plate (see FIG. 2).

A filling port 120a is formed in the cup bottom portion 210. To be more specific, in a state where the partition wall member 10a is disposed in an aortic duct inner space 825, the filling port 120a is disposed on a side of the cup bottom portion 210 on an aortic valve side 827.

The tapered portion 230 is formed such that an outer diameter from an outer peripheral edge 216 of the cup bottom portion 210 of the tapered portion 230 is increased in a direction toward a cup upper edge 220 having an approximately circular shape.

Although there may be the case where the tapered portion 230 functions as a partition wall body portion 100a, the tapered portion 230 basically functions as the seal portion 110. In other words, the tapered portion 230 in the embodiment 2 corresponds to the seal portion 110 in the embodiment 1.

A member per se which forms the tapered portion 230 (seal portion 110a) may be made of a resin. An outer side of the tapered portion 230 (seal portion 110a) may be covered by a resin-made member. As a resin, for example, a material such as silicon rubber can be adopted. Further, a surface of the tapered portion 230 may be formed into a shape (wrinkled shape) such that the surface of the tapered portion 230 easily bites into the aortic wall 822 or is easily caught by the aortic wall 822.

With such a configuration, adhesiveness between the aortic wall 822 of the aortic duct inner space 825 and the tapered portion 230 (seal portion 110a) can be further increased thus further increasing a sealing effect.

### (2) Additional Constitutional Elements

The aortic valve evaluation assisting jig 2 according to the embodiment 2 may also include additional constitutional elements described hereinafter in addition to the above-mentioned basic configuration.

### (a) Observation Window

An observation window 102 formed of a transparent member is disposed on the partition wall body portion 100a. The observation window 102 is provided for allowing a surgeon to view the aortic valve side 827 of the aortic duct inner space 825 from the space on the atmosphere open side 826 in a see-through manner in a state where the partition wall member 10a is disposed in the aortic duct inner space 825.

The observation window 102 may be formed of any member, and may have any shape and structure provided that the observation window 102 has the above-mentioned functions. In the example shown in FIG. 2A and FIG. 2B, the whole cup bottom portion 210 is formed of a transparent resin-made flat plate and hence, the cup bottom portion 210 also functions as the observation window 102. However, the observation window 102 is not limited to such a configuration, and may be formed using a member made of a different material, or only a portion of the cup bottom portion 210 is formed of a transparent member so as to use only the portion of the cup bottom portion 210 as the observation window.

### (b) Hollow Pipe 20, Grip 30, and Fluid System Connector 40

As shown in FIG. 2A and FIG. 2B, the aortic valve evaluation assisting jig 2 may further include a hollow pipe 20 in addition to the partition wall member 10a.

A liquid flow passage (not shown in the drawing) is formed in the hollow pipe 20, and an evaluation liquid 560 is made to flow through the liquid flow passage.

As the hollow pipe 20, for example, a member made of metal (for example, stainless steel such as SUS316), a member made of plastic or the like may be used. The hollow pipe 20 has an elongated shape as a whole. In the example shown in FIG. 2A and FIG. 2B, the hollow pipe 20 has a straight-line shape. However, the hollow pipe 20 is not limited to such a configuration, and the hollow pipe 20 may be bent at a middle portion of the hollow pipe 20, for example (also see an aortic valve evaluation assisting jig 3 according to the embodiment 3 described later).

One end of the hollow pipe 20 is inserted into a connecting port 132 formed in a connecting portion 130 of the partition wall member 10a and is stopped by a stopper 134, and is integrally formed with the partition wall member 10a. One end of a grip 30 is connected to the other end of the hollow pipe 20. A liquid flow passage is formed also in the grip 30 thus allowing an evaluation liquid 560 to flow therethrough. A fluid system connector 40 is disposed on the other end of the grip 30. For example, a three-way stopcock connector can be used as the fluid system connector 40 (see FIG. 2A and FIG. 2B).

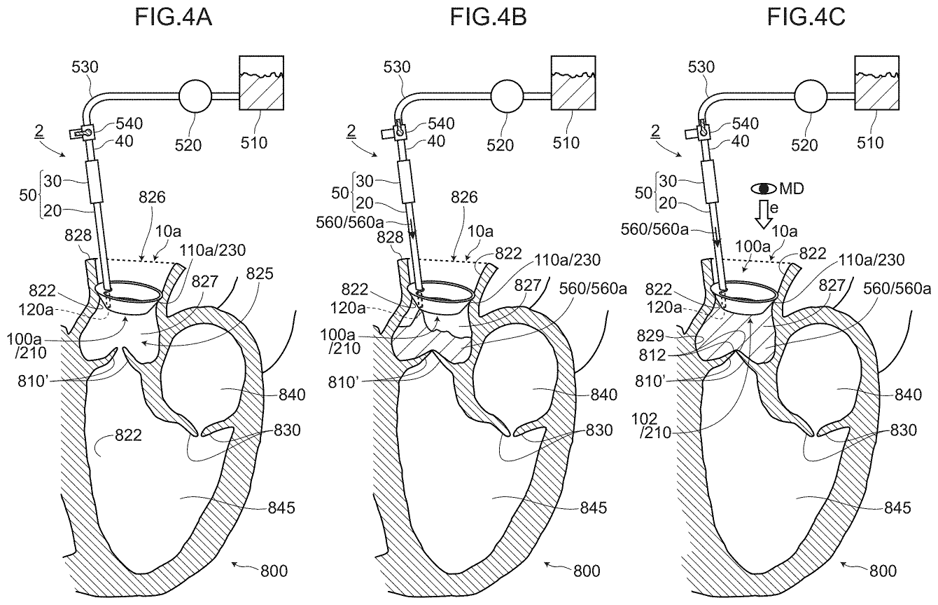

A liquid supply tube 530 is connected to the fluid system connector 40 (omitted in FIG. 2A, see FIG. 4C described later). The evaluation liquid 560 is supplied to and is made to flow through respective liquid flow passages formed in the fluid system connector 40, the grip 30, the hollow pipe 20, and the partition wall member 10a from the liquid supply tube 530, and the evaluation liquid 560 is eventually discharged from the filling port 120a formed in the partition wall member 10a.

The hollow pipe 20, the grip 30 and the like (possibly including other constitutional elements) form a handle 50.

In this embodiment, the description has been made on the premise that the grip 30 also has its own liquid flow passage. However, the grip 30 is not limited to such a configuration. For example, the inside of the grip 30 may be formed into a pipe shape, and the hollow pipe 20 may be inserted into the pipe-shaped inside of the grip 30. Further, the hollow pipe 20 may be configured to extend to the other end side of the grip 30 (in other words, the grip 30 being attached as a part of the hollow pipe 20).

One end of the hollow pipe 20 is arranged at the position where one end of the hollow pipe 20 does not become an obstacle in the arrangement of the above-mentioned observation window 102. That is, one end of the hollow pipe 20 is arranged at the position where one end of the hollow pipe 20 does not obstruct the observation of aortic valve 810′ by a surgeon.

### (3) Suitable Size

The partition wall member 10a in the embodiment 2 can adopt a suitable size corresponding to sizes and shapes of an aorta and an aortic valve of a patient. For example, in the case where the aortic valve evaluation assisting jig 2 is used for an adult patient, a diameter of a cup outer bottom surface 214 of the cup bottom portion 210 may be set to a value which falls approximately within a range of from 20 mm to 28 mm, for example. A diameter of the cup upper edge 220 may be set to a value which falls approximately within a range of from 35 mm to 45 mm and may preferably be set to approximately 40 mm. A length from the cup upper edge 220 to the cup inner bottom surface 212 which corresponds to the depth of the cup may be set to approximately 10 mm.

### 2. Use Example of Aortic Valve Evaluation Assisting Jig 2 According to Embodiment 2

FIG. 3A and FIG. 3B, and FIG. 4A to FIG. 4C are views for describing a use example of the aortic valve evaluation assisting jig 2 according to the embodiment 2. FIG. 3A and FIG. 3B, and FIG. 4A to FIG. 4C are views showing respective steps. In the drawings and the description, steps of aortic valvuloplasty are partially included.

### (1) Aorta Incision Step

Prior to the formation of the valve, a suitable portion of the aorta 820 (for example, incision portion “a” indicated by an arrow in FIG. 3A) is incised thus cutting the aorta 820. At this stage of the operation, the heart 800 is stopped (cardiac arrest). On the other hand, a portion of the aorta 820 downstream of the incision portion “a” is shut off by the aortic cross-clamp 550. That is, the forward flow and the backward flow of blood are shut off.

### (2) Valve Forming Step

When aorta is incised in aorta incision step, the inside of the duct (vessel) of the aorta (the aortic duct inner space 825) appears, and the aortic wall 822 and the aortic valve 810 (existing aortic valve) are open to the atmosphere. Symbol 828 indicates a cross section under such a state where the aortic wall 822 and the aortic valve 810 are open to the atmosphere. Suitable valve formation is performed corresponding to a state of a defect of the existing aortic valve 810 (see FIG. 3B, an arrow b showing one example of a portion where the valve formation is to be performed).

### (3) Arrangement Step of Aortic Valve Evaluation Assisting Jig 2

Next, at least a portion of the partition wall member 10a is disposed in the aortic duct inner space 825, and the aortic valve evaluation assisting jig 2 is disposed at predetermined portion in an operation field.

To be more specific, a surgeon holds the grip 30 of the aortic valve evaluation assisting jig 2 in his/her hand, arranges the posture of the aortic valve evaluation assisting jig 2 such that the filling port 120a faces the aortic valve 810′ side, and brings the seal portion 110a/the tapered portion 230 into contact with the aortic wall 822 of the aortic duct inner space side, and makes the partition wall member 10a wedge into the aortic duct inner space 825. At the stage of operation, the aortic wall 822, aortic valve commissure (not shown in the drawing) and the like which appear by incision are pulled out radially by a support suture so as to expand an opening portion of the cut surface 828 of the aorta so that a field of view of an aorta proximal portion (not shown in the drawing), the aortic valve 810 and the like can be favorably ensured.

On the other hand, the configuration is provided where the liquid supply tube 530 is connected to the fluid system connector 40. With such a configuration, the evaluation liquid 560 is supplied to the aortic valve evaluation assisting jig 2 through the liquid source 510, the liquid supplier 520 and a liquid supply tube 530 (see FIG. 4A).

### (4) Evaluation Liquid Charging Step

Next, while suitably adjusting an output of the liquid supplier 520, a cock of the fluid system connector 40 (three-way stopcock connector) is set in a flow direction. As a result, the evaluation liquid 560 is discharged from the filling port 120a. In this manner, the evaluation liquid 560 is filled in the space on an aortic valve side 827 in the aortic duct inner space 825 and hence, the space is filled with the evaluation liquid 560. At this point of time, assume that the evaluation liquid 560 is filled into the space while suitably releasing air through between the seal portion 110 and the aortic wall 822, for example (see FIG. 4B).

With respect to the formed aortic valve 810′, there may be the case where the aortic cusps 812 are not brought into contact with each other at the time of starting filling of the evaluation liquid 560. In such a case, it is possible to guide the aortic cusps 812 such that the aortic cusps 812 are brought into contact with each other by allowing a surgeon to change the flow of the evaluation liquid 560 by suitably changing an angle of the grip 30 and the hollow pipe 20 (collectively referred to as the handle 50) or by suitably changing a discharge pressure of the evaluation liquid 560 by suitably adjusting an angle of a cock of the fluid system connector 40 or an output of the liquid supplier 520.

### (5) Pressure Applying Step of Evaluation Liquid 560

Next, in a state where a sealing effect is obtained by bringing the seal portion 110a/the tapered portion 230 into close contact with the aortic wall 822, a filling pressure of the evaluation liquid 560 is suitably adjusted by suitably adjusting an angle of the cock of the fluid system connector 40 or an output of the liquid supplier 520, and a pressure of an intended magnitude is applied to the space in the aortic duct inner space 825 on an aortic valve side 827 (including Valsalva sinus 829).

With such an operation, a pressure (for example, a physiological pressure) of an intended magnitude of the evaluation liquid 560 which is a pressure in a reverse direction is applied to the aortic valve 810′ and hence, it is possible to bring the aortic cusps 812 of the aortic valve into contact with each other (see FIG. 4C).

The level of an intended pressure can be suitably set based on a state of a valve of the patient, an evaluation point of view or the like. For example, an intended pressure can be suitably set within a range of from approximately 50 mmHg to 150 mmHg.

### (6) Valve Observation and Evaluation Step

Next, in a state where a pressure is applied to the aortic valve 810′ in a reverse direction, the evaluation of the aortic valve 810′ is performed by observing the aortic valve 810′ (see FIG. 4C).

A surgeon MD can observe the aortic valve 810′ through the observation window 102 formed on the cup bottom portion 210 by viewing the aortic valve 810′ along an arrow e from the atmosphere open side 826, for example. The observation of the aortic valve 810′ is performed from a suitable point of view corresponding to the situation of the valve or the like. For example, viewpoints such as a contact state between the aortic cusps 812, the movement of the aortic valve 810′, a degree of inflation of Valsalva sinus 829, leakage of a liquid between the aortic cusps 812, lowering of a liquid level (water level) in the space on an aortic valve side 827 in the aortic duct inner space 825 may be confirmed.

### 3. Advantageous Effects Acquired by Aortic Valve Evaluation Assisting Jig 2 According to Embodiment 2

### (1) Tapered Portion 230 Having a Conical Side Surface Shape

The partition wall member 10a of the aortic valve evaluation assisting jig 2 has the tapered portion 230 formed such that an outer diameter of the tapered portion 230 is increased from the outer peripheral edge 216 of the cup bottom portion 210 toward the cup upper edge 220 having an approximately circular shape. A function of the seal portion 110a is imparted by such a tapered portion 230.

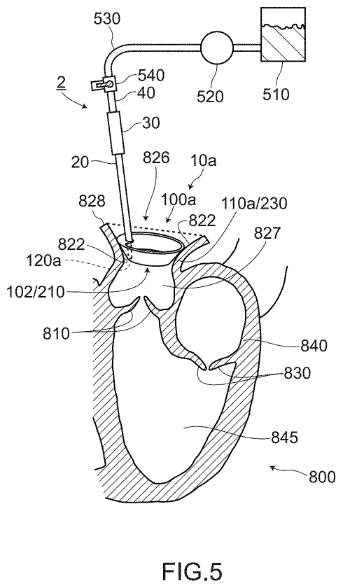

Accordingly, even when an inner diameter, a shape or the like of the aortic wall 822 of the aortic duct inner space 825 differs depending on a patient, the partition wall member 10a of the aortic valve evaluation assisting jig 2 has the tapered portion 230 and hence, the partition wall member 10a can be favorably fitted to the aortic walls 822 having various inner diameters, shapes or the like (for example, see FIG. 5 which is a view for describing a use example of the aortic valve evaluation assisting jig 2 according to the embodiment 2 (an example of another patient)).

### (2) Observation Window 102

The observation window 102 formed of a transparent member is disposed on the partition wall body portion 100a of the aortic valve evaluation assisting jig 2. The observation window 102 is provided for allowing a surgeon to view the space on the aortic valve side 827 in the aortic duct inner space 825 from the space on the atmosphere open side 826 in a see-through manner.

Since the observation window 102 is disposed in this manner, even in a state where a pressure is applied to the space on the aortic valve side 827 by the evaluation liquid 560, a surgeon can visually observe a mode of the aortic valve 810 or the like from the space on the atmosphere open side 826. A surgeon can view directly (direct viewing) from the space on the atmosphere open side 826 and hence, the surgeon can perform the evaluation of the valve with more certainty.

### (3) Elongated Handle 50

The hollow pipe 20 and the grip 30 form the elongated grip 50 as a whole. A surgeon can easily manipulate the aortic valve evaluation assisting jig 2 by grasping the grip 30 and by moving the handle including the grip 30. That is, the aortic valve evaluation assisting jig 2 has the handle 50 and hence, a surgeon can easily transmit a force from the handle 50 to the partition wall member 10a, and an angle of the partition wall member 10a can be also easily changed. Further, the elongated handle 50 can convert a manipulation amount of a surgeon at his/her hand into an extremely small displacement amount at the partition wall member 10a due to the principle of leverage and hence, even in an operation which requires a fine manipulation, the aortic valve evaluation assisting jig 2 can smoothly and reliably assist such an operation.

For example, the aortic valve evaluation assisting jig 2 has the handle 50 and hence, at the time of making the partition wall member 10a wedge into the aortic duct inner space 825 or at the time of pulling out the partition wall member 10a from the aortic duct inner space 825, the aortic valve evaluation assisting jig 2 can be easily manipulated. Further, for example, in the above-mentioned evaluation liquid charging step, when an angle at which the liquid is discharged is changed for changing the flow of the evaluation liquid 560, the aortic valve evaluation assisting jig 2 can be also easily manipulated. Still further, for example, in the above-mentioned pressure applying step of the evaluation liquid 560, a force can be easily transmitted to the partition wall member 10a against a pressure of the filled evaluation liquid 560. Still further, for example, in the above-mentioned valve observation and evaluation step, an angle of the observation window 102 (cup bottom portion 210) can be easily changed corresponding to the position of the part to be observed in a focusing manner.

The aortic valve evaluation assisting jig 2 according to the embodiment 2 has substantially the same configuration as the aortic valve evaluation assisting jig 1 according to the embodiment 1 except for the specific configuration of the partition wall member. Accordingly, the aortic valve evaluation assisting jig 2 according to the embodiment 2 acquires the corresponding advantageous effects found amongst all advantageous effects which the aortic valve evaluation assisting jig 1 according to the embodiment 1 acquires.

### Embodiment 3

### 1. Configuration of Aortic Valve Evaluation Assisting Jig 3 According to Embodiment 3

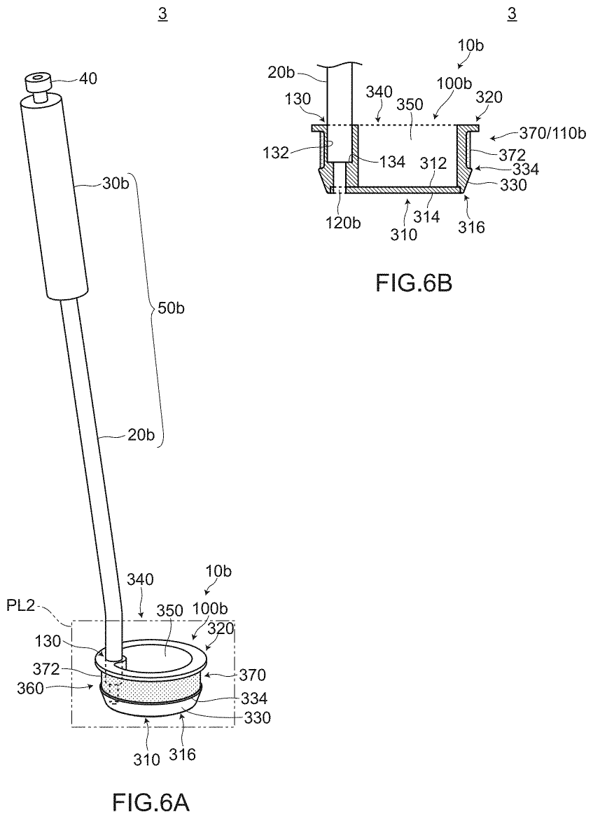

FIG. 6A and FIG. 6B are views for describing the aortic valve evaluation assisting jig 3 according to the embodiment 3. FIG. 6A is a perspective view of the aortic valve evaluation assisting jig 3, and FIG. 6B is a cross-sectional view of a main part of the aortic valve evaluation assisting jig 3 taken along an imaginary cross section PL2 in FIG. 6A. In the embodiment 3, with respect to constitutional elements having basically the same configurations and technical features as the corresponding constitutional elements of the embodiments 1, 2, the same symbols used in the embodiments 1, 2 are used, and the description of these constitutional elements is omitted.

The aortic valve evaluation assisting jig 3 according to the embodiment 3 basically has substantially the same configurations as the aortic valve evaluation assisting jigs 1, 2 according to the embodiments 1, 2. However, the aortic valve evaluation assisting jig 3 according to the embodiment 3 differs from the aortic valve evaluation assisting jigs 1, 2 according to the embodiments 1, 2 with respect to the specific configuration of the partition wall member.

### (1) Partition Wall Member 10b Having Approximately Circular Cylindrical Shape

As shown in FIG. 6A and FIG. 6B, the partition wall member 10b of the embodiment 3 has a bottomed approximately circular cylindrical shape. The partition wall member 10b has a cylindrical barrel portion 360 and a cylinder bottom portion 310. An opening portion 340 is formed on one side of the cylindrical barrel portion 360. The cylinder bottom portion 310 is connected to the other side of the cylindrical barrel portion 360. In this manner, the partition wall member 10b has the cylindrical barrel portion 360 and hence, as described later, it is possible to ensure a relatively large area where the partition wall member 10b is brought into contact with the aortic wall 822 of the aortic duct inner space side as described later.

The filling port 120b is formed in the cylinder bottom portion 310. To be more specific, the filling port 120b is formed on a side of the cylinder bottom portion 310 on the aortic valve side 827 when the partition wall member 10b is disposed in the aortic duct inner space 825.

### (2) Detail of Cylindrical Barrel Portion 360

A flange-shaped upper edge 320 is formed at the cylindrical barrel portion 360 on the opening portion 340 side.

A tapered portion 330 (a second tapered portion) and a groove portion 370 are formed on the cylindrical barrel portion 360. The tapered portion 330 has an outer diameter increasing from an outer peripheral edge 316 of the cylinder bottom portion 310 toward the opening portion 340 side. The groove portion 370 is disposed between an uppermost end 334 of the tapered portion 330 and the flange-shaped upper edge 320 and has a diameter smaller than an outer diameter of an uppermost end 334 of the tapered portion 330 and smaller than an outer diameter of the flange-shaped upper edge 320.

Although there may be the case where the groove portion 370 functions as the partition wall body portion 100b, the groove portion 370 basically functions as the seal portion 110. In other words, the groove portion 370 in the embodiment 3 corresponds to the seal portion 110 in the embodiment 1.

A member per se which forms the groove portion 370 (seal portion 110b) may be formed using a resin. Further, an outside of the groove portion 370 (seal portion 110b) may be covered by a resin-made member. For example, as shown in FIG. 6A and FIG. 6B, the groove portion 370 may be disposed such that a silicon rubber 372 is wrapped around the periphery of the groove portion 370.

With such a configuration, adhesiveness between the groove portion 370 (seal portion 110b) including the silicon rubber 372 and the aortic wall 822 can be further increased thus further increasing a sealing effect.

### (3) Bent-Shaped Handle 50b

The aortic valve evaluation assisting jig 3 adopts a bent-shaped hollow pipe 20b which is bent at a middle portion. Accordingly, the handle 50b of the aortic valve evaluation assisting jig 3 forms the bent-shaped handle which is bent at the middle portion as a whole (see FIG. 6A).

Other constitutional elements of the aortic valve evaluation assisting jig 3 have substantially the same configuration as the corresponding constitutional elements of the aortic valve evaluation assisting jig 2 according to the embodiment 2.

### 2. Advantageous Effects Obtained by Aortic Valve Evaluation Assisting Jig 3 According to Embodiment 3

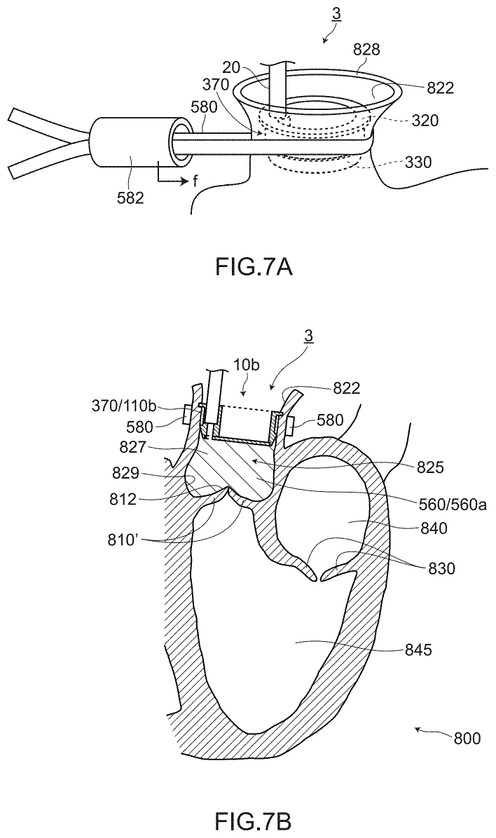

FIG. 7A and FIG. 7B are views for describing a use example of the aortic valve evaluation assisting jig 3 according to the embodiment 3. FIG. 7A is a perspective view of a main part showing a mode where the aortic wall 822 is strangulated from the outside. FIG. 7B is a cross-sectional view of the main part showing a mode in a state where the aortic wall 822 is strangulated from the outside in the same manner as FIG. 7A.

### (1) Strangulation of Aortic Wall 822 Using Groove Portion 370

The groove portion 370 is formed on the partition wall member 10b of the aortic valve evaluation assisting jig 3. Accordingly, prior to performing a pressure applying step of the evaluation liquid 560 (see the description of the embodiment 2), the aortic wall 822 can be strangulated using the groove portion 370.

For example, as shown in FIG. 7A, in a state where the partition wall member 10b is wedged into the aortic duct inner space 825, a strangulation tape 580 is wrapped around the aortic wall 822 from the outside of the aortic wall 822 at the position corresponding to the groove portion 370 and, thereafter, the aortic wall 822 is strangulated by moving the strangulation pipe 582 in a direction indicated by an arrow f. With such a configuration, as shown in FIG. 7B, in the aortic duct inner space 825, due to an action of strangulation, the aortic wall 822 is hermetically brought into pressure contact with the groove portion 370 at the position corresponding to the groove portion 370. Accordingly, adhesiveness between the groove portion 370 (seal portion 110b) and the aortic wall 822 can be further increased thus further increasing a sealing effect.

### (2) Tapered Portion 330

The tapered portion 330 which has an outer diameter increasing from an outer peripheral edge 316 of the cylinder bottom portion 310 toward the opening portion 340 side is formed on the cylindrical barrel portion 360 which forms a part of the partition wall member 10b (see FIG. 6A and FIG. 6B). The cylindrical barrel portion 360 has the tapered portion 330 whose outer diameter is gradually decreased toward a distal end and hence, the partition wall member 10b can be easily wedged into the aortic duct inner space 825.

### (3) Bent-Shaped Handle 50b

The handle 50b of the aortic valve evaluation assisting jig 3 is a bent-shaped handle which is bent at a middle portion as a whole. Accordingly, a grip 30 side of the handle 50b does not fall in a field of view of a surgeon and hence, the aortic value evaluation assisting jig 3 assists the surgeon to comfortably and smoothly perform an operation.

The aortic valve evaluation assisting jig 3 according to the embodiment 3 has substantially the same configuration as the aortic valve evaluation assisting jigs 1, 2 according to the embodiments 1, 2 except for the specific configuration of the partition wall member. Accordingly, the aortic valve evaluation assisting jig 3 according to the embodiment 3 acquires the corresponding advantageous effects found amongst all advantageous effects which the aortic valve evaluation assisting jigs 1, 2 according to the embodiments 1, 2 acquire.

### Modification

Although the present invention has been described based on the above-mentioned embodiments, the present invention is not limited to the above-mentioned embodiments, and the present invention can be carried out in various modes without departing from the gist of the present invention, for example, the following modifications are also conceivable.

(1) The numbers, the materials, the shapes, the positions, the sizes and the like of the constitutional elements described in the above-mentioned embodiments are provided only for an exemplifying purpose, and these can be changed within ranges where advantageous effects of the present invention are not impaired.
(2) The aortic valve evaluation assisting jigs 2, 3 of the embodiments 2, 3 are described by estimating the case where the aortic valve evaluation assisting jig 2, 3 has the observation window 102, and the observation of the aortic valve 810′ is performed only by direct viewing (direct viewing). However, the present invention is not limited to such observation.
(a) The movement of the valve, the flow of blood and the like may be indirectly observed from the outside of the heart 800 by ultrasonic diagnostic equipment (echocardiography) in place of direct viewing or together with direct viewing, for example.
(b) Further, the observation of the valve may be performed by viewing using a camera 104 disposed on the partition wall body portion in place of direct viewing or together with direct viewing, for example.

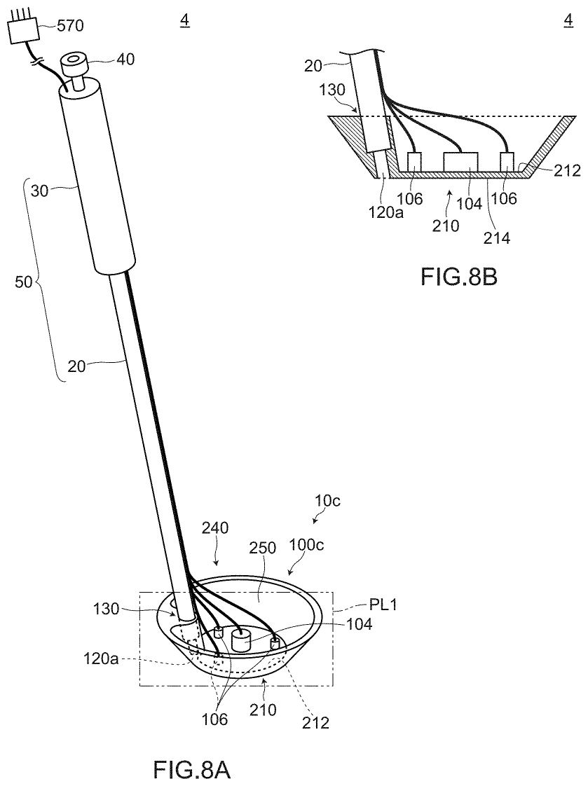

FIG. 8A and FIG. 8B are views for describing an aortic valve evaluation assisting jig 4 according to a modification.

As shown in FIG. 8A and FIG. 8B, the camera 104 which images a mode of the space on the aortic valve side 827 of the aortic duct inner space 825 may be disposed on the partition wall body portion 100c (to be more specific, the cup bottom portion 210 or the cylinder bottom portion 310). As the camera 104, for example, a charge coupled device (CCD) camera with an ocular lens can be used, for example.

It is further preferable that the partition wall body portion 100c (the cup bottom portion 210 or the cylinder bottom portion 310) have a light projecting device 106 which projects light to the aortic valve side 827 of the aortic duct inner space 825. As the light projecting device 106, a light emitting diode (LED) light source can be used, for example.

Cables (not indicated by symbols) are respectively connected to the camera 104 and the light projecting device 106 disposed on the partition wall member 10. The cables form transmission paths for electricity and signals relating to the camera 104 and the light projecting device 106. The cables are wired along the longitudinal direction of the handle 50, and are connected to an electric system connector 570 on a side of the handle 50 opposite to a side where the partition wall member 10 is mounted on the handle 50.

When a surgeon observes the aortic valve 810′ by direct viewing through the observation window 102, there may be the case where light of a head lamp which the surgeon wears is reflected on a surface of the observation window 102 so that the surgeon has difficulty in observing the aortic valve side 827. Even in such a case, with the provision of the camera 104, the surgeon can observe a mode in the space on the aortic valve side 827 through the camera 104 without being affected by a light of the head lamp.

Further, with the provision of the light projecting device 106, even in the case where the illuminance in the space on the aortic valve side 827 is low, the light projecting device 106 projects light into the space and hence, it is possible to illuminate the inside of the space more clearly. Accordingly, the surgeon can observe the mode in the space with a finer image and hence, more accurate valve observation can be performed.

(3) The cup bottom portion 210 of the embodiment 2 and the cylinder bottom portion 310 of the embodiment 3 have been described by taking the case where these members are formed of a disk like flat plate as an example. However, the present invention is not limited to such an example.

For example, the cup bottom portion 210 or the cylinder bottom portion 310 may be formed of an angular flat plate (not shown in the drawing).

Further, the cup bottom portion 210 or the cylinder bottom portion 310 may not be formed in a completely flat shape. For example, the cup bottom portion 210 or the cylinder bottom portion 310 may be formed in a concave shape or a convex shape so that the cup bottom portion 210 or the cylinder bottom portion 310 exhibits a function as a lens, for example (not shown in the drawing). The cup bottom portion and the cylinder bottom portion having such configurations also fall within a range of equivalents of the cup bottom portion 210 of the embodiment 2 and the cylinder bottom portion 310 of the embodiment 3.

(4) With respect to the groove portion 370 of the embodiment 3, the configuration is exemplified in FIG. 6 where the groove portion 370 is formed parallel to the inner wall of the cylindrical barrel portion 360. However, the present invention is not limited to such an example.

For example, as in the case of the tapered portion 230 of the aortic valve evaluation assisting jig 2, the groove portion 370 may be formed in a tapered shape such that an outer diameter of the groove portion 370 is increased from a cylinder bottom portion 310 side toward an upper edge 320 in a flange shape.

(5) In the above-mentioned respective embodiments, the description has been made by estimating the case where, in evaluation liquid charging step (see the description of embodiment 2), air which is present in the space of aortic duct inner space 825 on the aortic valve side 827 is suitably released through between the seal portions 110, 110a, 110b and the aortic wall 822. However, the present invention is not limited to such a case.

For example, an air release port (not shown in the drawing) may be formed in the partition wall member of the aortic valve evaluation assisting jig 1, 2, 3, 4. The air release port is configured such that a filter member having property of allowing air to pass therethrough and preventing the evaluation liquid 560 from passing therethrough is embedded in the air release port, and air in the space on the aortic valve side 827 in the aortic duct inner space 825 can be released through the air release port.

With such a configuration, the evaluation liquid 560 can be smoothly filled in the space of the aortic duct inner space 825 on the aortic valve side 827.

(6) In the above-mentioned respective embodiments, a pressure monitor for monitoring a pressure of the evaluation liquid 560 filled in the space on the aortic valve side 827 in the aortic duct inner space 825 may be disposed on the partition wall member.

With such a configuration, it is possible to evaluate the valve while ensuring whether or not an intended pressure (intended physiological pressure) can be applied with certainty.

What is claimed is:

1. An aortic valve evaluation assisting jig which assists evaluation of an aortic valve formed by aortic valvuloplasty at the time of performing the evaluation of the aortic valve, wherein the aortic valve evaluation assisting jig comprises a partition wall member which partitions an aortic duct inner space into a space on an atmosphere open side and a space on an aortic valve side in a state where at least a part of the partition wall member is disposed in the aortic duct inner space, and
the partition wall member has: a partition wall body portion;
a seal portion which seals an evaluation liquid in a state where the seal portion is brought into contact with the aortic wall of an aortic duct inner space side; and
a filling port through which the evaluation liquid is filled into the space on the aortic valve side in the aortic duct inner space.

2. The aortic valve evaluation assisting jig according to claim 1, wherein an observation window formed of a transparent member through which the space on the aortic valve side in the aortic duct inner space can be viewed from the space on the atmosphere open side in a see-through manner is disposed on the partition wall body portion.

3. The aortic valve evaluation assisting jig according to claim 1, wherein a camera which images a mode of the space on the aortic valve side in the aortic duct inner space is disposed on the partition wall body portion.

4. The aortic valve evaluation assisting jig according to claim 1, wherein the partition wall member is formed in an approximately cup shape and is formed of: a cup bottom portion having a flat plate-like shape; and a tapered portion formed such that an outer diameter is increased from an outer peripheral edge of the cup bottom portion toward a cup upper edge having an approximately circular shape,
the filling port is formed in the cup bottom portion, and
the seal portion is formed of the tapered portion.

5. The aortic valve evaluation assisting jig according to claim 1, wherein the partition wall member is formed in a bottomed approximately cylindrical shape and is formed of: a cylindrical barrel portion having an opening portion on one side; and a cylinder bottom portion connected to the other side of the cylindrical barrel portion, wherein
a flange-shaped upper edge is formed at the cylindrical barrel portion on an opening portion side,
the cylindrical barrel portion has: a tapered portion which has an outer diameter increasing from an outer peripheral edge of the cylinder bottom portion toward the opening portion side; and a groove portion which is disposed between an uppermost end of the tapered portion and the flange-shaped upper edge and has a diameter smaller than an outer diameter of the uppermost end of the tapered portion and smaller than an outer diameter of the flange-shaped upper edge,
the filling port is formed in the cylinder bottom portion, and
the seal portion is formed of the groove portion.

6. The aortic valve evaluation assisting jig according to claim 4, further comprises a hollow pipe in which a liquid flow passage is formed, and
one end of the hollow pipe is connected to the filling port formed in the partition wall member.

7. The aortic valve evaluation assisting jig according to claim 1, wherein the seal portion is formed using a resin, or an outside of the seal portion is covered by a resin-made member.
